(19) United States
(12) Patent Application Publication
(10) Pub. No.: US 2008/0118554 A1
(43) Pub. Date: May 22, 2008

(54) Pharmaceutical compositions comprising a combination of piperidinoalkanol and decongestant

(76) Inventors: Limor Ari-Pardo, Beit Halevi (IL); Sivan Antler, Modiin (IL); Rina Zilberman, Kfar-Sava (IL)

Correspondence Address: KENYON & KENYON LLP, ONE BROADWAY, NEW YORK, NY, 10004, US

(21) Appl. No.: 11/824,537

(22) Filed: Jun. 28, 2007

Related U.S. Application Data
(60) Provisional application No. 60/817,411, filed on Jun. 30, 2006.

(51) Int. Cl. A61K 9/48; A61K 9/26; A61K 31/445

(52) U.S. Cl. 424/451; 424/469; 514/317

(57) ABSTRACT

A pharmaceutical composition comprising a first therapeutic agent and a second therapeutic agent is described. A plurality of particles comprising (i) an interior comprising the second therapeutic agent and (ii) an exterior comprising a material for controlling the release of the second therapeutic agent may be disposed within a mixture, wherein the mixture comprises the first therapeutic agent. The first therapeutic agent may be a piperidinoalkanol, such as fexofenadine, and the second therapeutic agent may be a decongestant, such as pseudoephedrine. The interior may comprise an inner core and an intermediate layer disposed over the inner core, wherein the second therapeutic agent is contained in the intermediate layer. Methods of treating congestion with a pharmaceutical composition comprising a piperidinoalkanol and a decongestant are also described.

### CROSS-REFERENCE TO RELATED APPLICATIONS

This application claims the benefit of U.S. Provisional Application Ser. No. 60/817,411, filed on Jun. 30, 2006, which is incorporated by reference herein.

### TECHNICAL FIELD

This invention relates to pharmaceutical compositions comprising a combination of a piperidinoalkanol and a decongestant.

### BACKGROUND

Piperidinoalkanol compounds (e.g., fexofenadine) and decongestants (e.g., pseudoephedrine) are drugs that are commonly used in the treatment of nasal congestion, which can result from various disorders such as allergic rhinitis (nasal allergies). The combination of a piperidinoalkanol and a decongestant can be more effective than either alone in the treatment of nasal congestion.

U.S. Pat. No. 6,613,357 (Faour et al.) describes an osmotic device containing controlled release pseudoephedrine in the core in combination with a rapid release H1 antagonist in an external coat. U.S. Pat. No. 6,039,974 (MacLaren et al.) describes a combination of piperidinoalkanol and decongestant in the form of a bilayer tablet. U.S. Pat. No. 6,004,582 (Faour et al.) describes a multi-layered osmotic device. U.S. Pat. No. 6,537,573 (Johnson et al.), which is incorporated by reference herein, discloses a dosage form containing cetirizine as an intermediate release component and pseudoephedrine as a controlled release component.

Without controlled release of the drugs, the schedule for administering a combination of fexofenadine and decongestant is typically four doses per day. In order to provide a once-daily or twice-daily dosage form, for example, a formulation providing a relatively immediate release of the piperidinoalkanol with an extended release of the decongestant is desirable.

### SUMMARY OF THE INVENTION

In one aspect, the present invention provides a pharmaceutical composition comprising: (a) a mixture comprising a first therapeutic agent; and (b) a plurality of particles disposed within the mixture, wherein the particles comprise (i) an interior comprising a second therapeutic agent, and (ii) an exterior comprising a material for controlling the release of the second therapeutic agent. In some embodiments, the first therapeutic agent comprises a piperidinoalkanol and the second therapeutic agent comprises a decongestant.

In another aspect, the present invention provides a method for making a pharmaceutical composition, comprising the steps of: (a) forming a plurality of particles, comprising the steps of (i) providing an inner core, (ii) disposing an intermediate layer containing a second therapeutic agent over the inner core, and (iii) disposing an extended release layer over the intermediate layer, wherein the extended release layer comprises a material for controlling the release of the second therapeutic agent; and (b) combining the particles with a mixture comprising a first therapeutic agent.

In another aspect, the present invention provides a method for making a pharmaceutical composition, comprising the step of combining: (a) a plurality of particles comprising (i) an interior comprising a second therapeutic agent; and (ii) an exterior comprising a material for controlling the release of the second therapeutic agent; and (b) a mixture comprising a first therapeutic agent.

In another aspect, the present invention provides a method of treating congestion in a patient in need thereof by administering to the patient, a pharmaceutical composition comprising: (a) a mixture comprising a piperidinoalkanol; and (b) a plurality of particles disposed within the mixture, wherein the particles comprise (i) an interior comprising a decongestant, and (ii) an exterior comprising a material for controlling the release of the decongestant.

In another aspect, the present invention provides the use of a piperidinoalkanol and a decongestant in the manufacture of a medicament for the treatment of congestion, wherein the medicament comprises: (a) a mixture comprising a piperidinoalkanol; and (b) a plurality of particles disposed within the mixture, wherein the particles comprise (i) an interior comprising a decongestant, and (ii) an exterior comprising a material for controlling the release of the decongestant.

### BRIEF DESCRIPTION OF THE DRAWINGS

FIG. 1 shows the pseudoephedrine HCl dissolution profiles of formulation Examples 8 and 7.

FIG. 2 shows the pseudoephedrine HCl dissolution profiles of formulation Examples 10 and 7.

FIG. 3 shows the fexofenadine HCl dissolution profiles of formulation Examples 14 and 17.

FIG. 4 shows the fexofenadine HCl dissolution profiles of formulation Examples 16 and 17.

FIG. 5A shows the fexofenadine HCl dissolution profiles of formulation Examples 17 and 18.

FIG. 5B shows the fexofenadine HCl dissolution profiles of formulation Examples 19 and 20.

FIG. 5C shows the fexofenadine HCl dissolution profiles of formulation Examples 17 and 19.

FIG. 6A shows the pseudoephedrine HCl dissolution profiles of formulation Example 17 in various solutions.

FIG. 6B shows the fexofenadine HCl dissolution profiles of formulation Example 17 in various solutions.

### DETAILED DESCRIPTION

In one aspect, the present invention provides a pharmaceutical composition comprising a mixture of a first therapeutic agent and at least one pharmaceutically acceptable excipient. The mixture may be formed using any of various pharmaceutical manufacturing processes, including direct compression, dry granulation, wet granulation, or pelletization. Preferably, the mixture is a dry blend formed by dry granulation or direct compression.

In preferred embodiments, the first therapeutic agent is an antihistamine, which is preferably an H1 antagonist, and more preferably a member of the piperidinoalkanol class of compounds, which includes, for example, fexofenadine, loratadine, cetirizine, terfenadine, acrivastine, astemizole, and pharmaceutically acceptable salts thereof. Various types of pharmaceutically acceptable excipients are suitable for use in the mixture, including binders, fillers, film coating polymers, plasticizers, glidants, disintegrants, lubricants, etc. Preferably, the mixture is formulated to allow for the immediate release of the first therapeutic agent.

The pharmaceutical composition further comprises a plurality of particles disposed within the mixture, wherein the particles contain a second therapeutic agent. The particles preferably have an interior containing the second therapeutic agent and an exterior containing a material for controlling the release of the second therapeutic agent. In preferred embodiments, the second therapeutic agent is a decongestant, which may be any decongestant known in the art, such as pseudoephedrine, that can be used to reduce congestion in the upper respiratory tract.

In a preferred embodiment, the particles comprise an interior and an exterior. Preferably, the interior comprises an inner core and an intermediate layer disposed between the inner core and the exterior. Preferably, the exterior comprises an extended release layer. The term “inner core” as used herein refers to a core for carrying a pharmaceutical formulation that is preferably both inert and non-toxic. The inner core preferably comprises a pharmaceutically inactive material, such as microcrystalline cellulose spheres (e.g., Cellets®) or sugar spheres. In some cases, the inner core is a granulated core comprising a decongestant and any pharmaceutically acceptable excipient. In some cases, preferably at least 85% of the cores are 100-1000 μm in size; and in some cases, 100-850 μm in size; and in some cases, 100-710 μm in size; and in some cases, 100-500 μm in size; and in some cases, 100-425 μm in size; and in some cases, 100-355 μm in size; and in some cases, 200-355 μm in size.

The intermediate layer preferably comprises a decongestant, such as pseudoephedrine HCl. Preferably, the intermediate layer may further comprise at least one pharmaceutically acceptable binder such as polyvinyl pyrrolidone (PVP), methyl cellulose, hydroxypropyl cellulose, or hydroxypropyl methylcellulose. In some cases, use of PVP as a binder is preferred. The intermediate layer may be applied directly onto the inner core or it may be applied to one or more layers or coatings on the inner core. The intermediate layer may be applied in various ways, including for example, by spray coating a solution containing a suitable solvent and the decongestant. The solvent may be any of various solvents typically used in the art, including for example, an alcohol, water, isopropanol, acetone, or mixtures thereof. The spray coating process may be performed in any of various ways, including for example, by using a fluid bed drier equipped with a Wurster column and bottom spray nozzle.

The extended release layer preferably comprises a material for controlling the release of the decongestant. Such materials are known in the art and preferably, the material for the extended release layer is a polymeric material. Examples of suitable polymeric materials include hydroxypropyl cellulose, hydroxypropyl methylcellulose, ethyl cellulose, and polymethacrylates. The extended release layer may further comprise one or more plasticizers. Preferably, the plasticizers have hydrophilic and hydrophobic qualities. The plasticizers may differ from each other in their degree of solubility, hydrophobicity, and/or hydrophilicity. Examples of plasticizers suitable for use in the present invention include triethyl citrate, polyethylene glycol, diethyl phthalate, dibutyl phthalate, dibutyl sebecate, and acetyl tributyl citrate. The extended release layer may be applied onto the intermediate layer in any of various ways, including, for example, by spray coating.

In some embodiments, the intermediate layer is completely encapsulated within the extended release layer. The extended release layer may be porous to fluid and drug. As such, the mechanism for controlled release of the decongestant may be by diffusion through the extended release layer.

Any part of the pharmaceutical composition may further comprise any pharmaceutically acceptable excipient such as binders, film coating polymers, plasticizers, glidants, disintegrants, lubricants, etc. In preferred embodiments, the pharmaceutical composition is an oral dosage form. For example, the composition may be compressed into a tablet or filled into a capsule.

In certain embodiments, the oral dosage form can be administered once or twice daily. Preferably, the decongestant is released in an extended-release fashion. As used herein, the term “extended-release” refers to the release of the active material content that allows for once or twice-daily dosing of the oral formulation. In some cases, less than about 70% of the decongestant is released in a time period of 8 hours after exposure of the oral dosage form to an aqueous solution; and in some cases, less than 50% under the same conditions. The antihistamine is preferably released in an immediate-release fashion. As used herein, the term “immediate-release” refers to the release of the majority of the active material content within a relatively short time after oral ingestion. In some cases, at least 50% of the piperidinoalkanol is released within 15 minutes after exposure of the oral dosage form to an aqueous solution; and in some cases, at least 75% under the same conditions.

The spatial distribution of the particles will vary according to the particular application. In preferred embodiments, the spatial distribution of the particles are substantially uniform with little or no agglomeration of the particles. Particle size ranges and size distributions will vary according to the particular application. In preferred embodiments, at least 85% of the particles have a size in the range of about 425 to about 600 μm.

Another aspect of the present invention provides a method for treating a patient's congestion of the upper respiratory tract, such as nasal congestion. Nasal congestion can arise from various conditions, including an allergy-related disorder, such as allergic rhinitis. The method comprises the step of administering a pharmaceutical composition of the present invention to a patient. In certain embodiments, the pharmaceutical composition may be administered once or twice daily. The pharmaceutical composition may be administered orally, as a tablet or capsule for example.

Another aspect of the present invention provides a method for making a pharmaceutical composition having a combination of a piperidinoalkanol and a decongestant. In a preferred embodiment, a plurality of particles are formed by providing an inner core, forming an intermediate layer containing a decongestant over the inner core, and forming an extended release layer over the intermediate layer, wherein the extended release layer comprises a material for controlling the release of the decongestant. The plurality of particles are combined with a mixture comprising a piperidinoalkanol. The composition may then be compressed into a tablet or used to fill a capsule. The various layers may be formed by, for example, spray coating using techniques known in the art.

An exemplary method for making the pharmaceutical composition is as follows:

Step 1: Cellets® (microcrystalline cellulose) are coated with a hydro-alcoholic solution (e.g., 95% ethanol:water in a 1:2 ratio) containing pseudoephedrine HCl and polyvinyl pyrrolidone (PVP K-30). The solution is applied onto the Cellets using a fluid bed drier equipped with a Wurster column (bottom spray). This step results in the formation of an intermediate drug layer over the Cellets.

Step 2: A film-coating polymer (e.g., ethylcellulose) is dissolved in a suitable solvent (e.g., acetone:95% ethanol mixture in a 1:1.25 ratio). Next, a hydrophilic plasticizer (e.g., polyethylene glycol (PEG)) and a hydrophobic plasticizer (e.g., dibutyl sebacate (DBS)) are added. Next, water is added and the solution is mixed until homogeneous. Next, the solution is sprayed using a fluid bed drier equipped with a Wurster Column (bottom spray). This step results in the formation of an extended release layer over the intermediate drug layer.

Step 3: The decongestant-containing particles resulting from Step 2 are mixed with a piperidinoalkanol, such as fexofenadine, along with excipients such as glidants, fillers, disintegrants, or lubricants. The composition is then compressed into tablets, filled into capsules, or the like.

### FORMULATION EXAMPLES

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

The following examples are given for the purpose of illustrating the invention and shall not be construed as limiting the scope or spirit of the invention.

Table A below shows the composition of particle formulation Examples 1 through 4. Each of the examples has a different composition for the intermediate layer. All concentrations are provided as weight %.

In preferred embodiments, a higher solution concentration (solid concentration by wt %) can be used to improve the morphology and/or uniformity of the intermediate drug layer. Furthermore, a higher solution concentration can provide improved process yield (in some cases, above 95%) and improved weight gains (in some cases, above 90%). Furthermore, a higher solution concentration can shorten the process time. Therefore, Example 4 exemplifies a preferred embodiment, where the decongestant layer is formed using a solution concentration of 60%.

TABLE A
Intermediate Drug Layer (Pseudoephedrine) Formulations

Formulation Example

| | | | | |
|---|---|---|---|---|
| Materials | 1 | 2 | 3 | 4 |

Core

| | | | | |
|---|---|---|---|---|
| Cellets 200-355 μm | 120 | 120 | 90 | 120 |

Intermediate Layer

| | | | | |
|---|---|---|---|---|
| PVP K-30 | 14 | 20 | 14 | 14 |
| Pseudoephedrine HCl | 240 | 240 | 240 | 240 |
| Solution Concentration | 20% | 25% | 30% | 60% |
| Total Intermediate Layer | 374 | 380 | 344 | 374 |
| Weight (mg) | | | | |

Table B shows the composition of particle formulation Examples 5 through 12. Solvent A is water:isopropanol:ethanol in a 4:5:10 ratio. Solvent B is isopropanol:acetone in a 1:2 ratio. Solvent C is water:ethanol:acetone in a 1:4:5 ratio. In the row indicating the ratio between hydrophobic:hydrophilic plasticizer, the notation “1:0” is meant to indicate that only hydrophobic plasticizer is used. Each of Examples 5 through 12 has a different composition for the extended release layer.

TABLE B
Extended Release Layer Formulations

| | Formulation Example | | | | | | | |
|---|---|---|---|---|---|---|---|---|
| Materials | 5 | 6 | 7 | 8 | 9 | 10 | 11 | 12 |

Core

| | | | | | | | | |
|---|---|---|---|---|---|---|---|---|
| Cellets 200-355 μm | 120 | 120 | 120 | 120 | 120 | 120 | 90 | 90 |

Intermediate Layer

| | | | | | | | | |
|---|---|---|---|---|---|---|---|---|
| PVP K-30 | 14 | 14 | 14 | 14 | 14 | 14 | 14 | 14 |
| Pseudoephedrine | 240 | 240 | 240 | 240 | 240 | 240 | 240 | 240 |
| HCl | | | | | | | | |
| Total Intermediate | 374 | 374 | 374 | 374 | 374 | 374 | 344 | 344 |
| Layer Weight (mg) | | | | | | | | |

Extended Release Layer

| | | | | | | | | |
|---|---|---|---|---|---|---|---|---|
| Ethocel 7 cps | 50 | — | | | | | | |
| Ethocel 100 cps | — | 170 | 272 | 272 | 180 | 240 | 164 | 220 |
| Dibutyl Sebacate (DBS) | 7 | 17 | 32 | 8 | 22 | 30 | 21 | 27.5 |
| Polyethylene | 7 | — | — | — | 22 | 30 | 21 | 27.5 |
| Glycol 400 | | | | | | | | |
| Solvents | A | B | B | B | C | C | C | C |
| Ratio between | 1:1 | 1:0 | 1:0 | 1:0 | 1:1 | 1:1 | 1:1 | 1:1 |
| hydrophobic:hydro- | | | | | | | | |
| philic plasticizer | | | | | | | | |
| Total Extended | 438 | 561 | 678 | 654 | 598 | 674 | 550 | 619 |
| Release Layer | | | | | | | | |
| Weight (mg) | | | | | | | | |

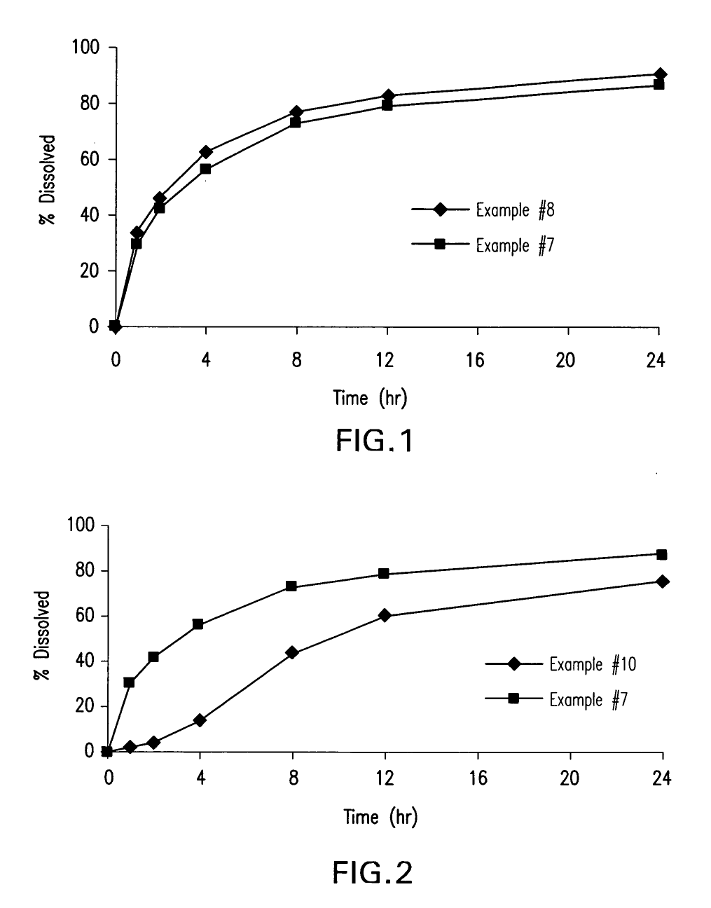

Table 1 below shows the pseudoephedrine HCl dissolution profiles of particle formulation Examples 8 and 7 (RSD indicates relative standard deviation), which is plotted in FIG. 1. All dissolution profiles provided herein were obtained in a 0.001N HCl solution (unless otherwise indicated) using a USP Type II dissolution apparatus that was equipped with a paddle stirring at 50 rpm and at 37° C. Example 7 contained 32 mg dibutyl sebacate (DBS) and Example 8 contained 8 mg DBS. These results indicate that the amount of DBS in the extended release layer does not significantly affect the pseudoephedrine HCl dissolution profile. If a reduction in the pseudoephedrine HCl dissolution rate is desired, a hydrophilic plasticizer (such as polyethylene glycol 400) for example, may be added to the extended release coating layer.

TABLE 1
% Dissolution of Pseudoephedrine HCl

| Time | Example | | Example | |
|---|---|---|---|---|
| (hr) | 8 | RSD | 7 | RSD |
| 0 | 0 | | 0 | 0 |
| 1 | 34 | 22.8 | 30 | 17.0 |
| 2 | 47 | 16.3 | 42 | 11.5 |
| 4 | 63 | 10.8 | 56 | 8.1 |
| 8 | 77 | 7.2 | 73 | 4.7 |
| 12 | 83 | 5.7 | 79 | 3.6 |
| 24 | 90 | 4.6 | 87 | 3.1 |

Table 2 below shows the pseudoephedrine HCl dissolution profiles of particle formulation Examples 10 and 7, which is plotted in FIG. 2. Example 10 has a hydrophobic:hydrophilic plasticizer ratio of 1:1 in the extended release layer, whereas Example 7 contains only hydrophobic plasticizer. These results demonstrate that adding a hydrophilic plasticizer to the extended release layer can reduce the dissolution rate of the pseudoephedrine HCl. Where a hydrophilic plasticizer was added to the formulation, such as in Example 10, an aqueous solvent was used, such as water:95% ethanol:acetone in a 1:4:5 ratio.

TABLE 2
% Dissolution of Pseudoephedrine HCl

| Time | Example | | Example | |
|---|---|---|---|---|
| (hr) | 10 | RSD | 7 | RSD |
| 0 | 0 | 0 | 0 | 0 |
| 1 | 2 | 5.3 | 30 | 17.0 |
| 2 | 4 | 4.1 | 42 | 11.5 |
| 4 | 14 | 0.9 | 56 | 8.1 |
| 8 | 44 | 1.0 | 73 | 4.7 |
| 12 | 60 | 1.1 | 79 | 3.6 |
| 24 | 76 | 1.0 | 87 | 3.1 |

Table C below shows the composition of tablet formulation Examples 13 through 20. Each of the examples has a different composition for the mixture of an antihistamine and at least one pharmaceutically acceptable excipient.

TABLE C
Tablet Formulations

| | Formulation Example | | | | | | | |
|---|---|---|---|---|---|---|---|---|
| Materials | 13 | 14 | 15 | 16 | 17 | 18 | 19 | 20 |

Core

| | | | | | | | | |
|---|---|---|---|---|---|---|---|---|
| Cellets 200-355 μm | 120 | 120 | 120 | 120 | 120 | 90 | 120 | 120 |

Intermediate Layer

| | | | | | | | | |
|---|---|---|---|---|---|---|---|---|
| PVP K-30 | 14 | 14 | 14 | 14 | 14 | 14 | 14 | 14 |
| Pseudoephedrine HCl | 240 | 240 | 240 | 240 | 240 | 240 | 240 | 240 |
| Total Intermediate | 374 | 374 | 374 | 374 | 374 | 344 | 374 | 374 |
| Layer Weight (mg) | | | | | | | | |

Extended Release Layer

| | | | | | | | | |
|---|---|---|---|---|---|---|---|---|
| Ethocel 100 cps | 241 | 241 | 241 | 241 | 241 | 220 | 240 | 240 |
| Dibutyl Sebacate | 29 | 29 | 29 | 29 | 29 | 27.5 | 30 | 30 |
| PEG 400 | 30 | 30 | 30 | 30 | 30 | 27.5 | 30 | 30 |
| Total Extended Release | 674 | 674 | 674 | 674 | 674 | 619 | 674 | 674 |
| Layer Weight (mg) | | | | | | | | |

Mixture

| | | | | | | | | |
|---|---|---|---|---|---|---|---|---|
| Syloid 244 | 74 | 74 | 74 | 74 | 74 | 68 | 74 | 74 |
| Avicel PH101 | 128 | 156 | 133 | 128 | 156 | | 133 | |
| Mannitol Parteck | | | | | | 132 | | |
| Lactose Spray Dried | | | | | | | | 105 |
| Fexofenadine HCl | 180 | 180 | 180 | 180 | 180 | 180 | 180 | 180 |
| Crospovidone | 56 | 28 | | | | | | |
| Sodium Starch Glycolate | | | | 56 | 28 | 26 | 28 | 56 |
| Starch 1500 | | | 56 | | | | | |
| Sodium Lauryl Sulfate | | | | | | | 22 | 22 |
| Magnesium Stearate | 8 | 8 | 3 | 8 | 8 | 7 | 9 | 9 |
| % Disintegrant | 5% | 2.5% | 5% | 5% | 2.5% | 2.5% | 2.5% | 5% |
| Total Tablet Weight (mg) | 1120 | 1120 | 1120 | 1120 | 1120 | 1032 | 1120 | 1120 |

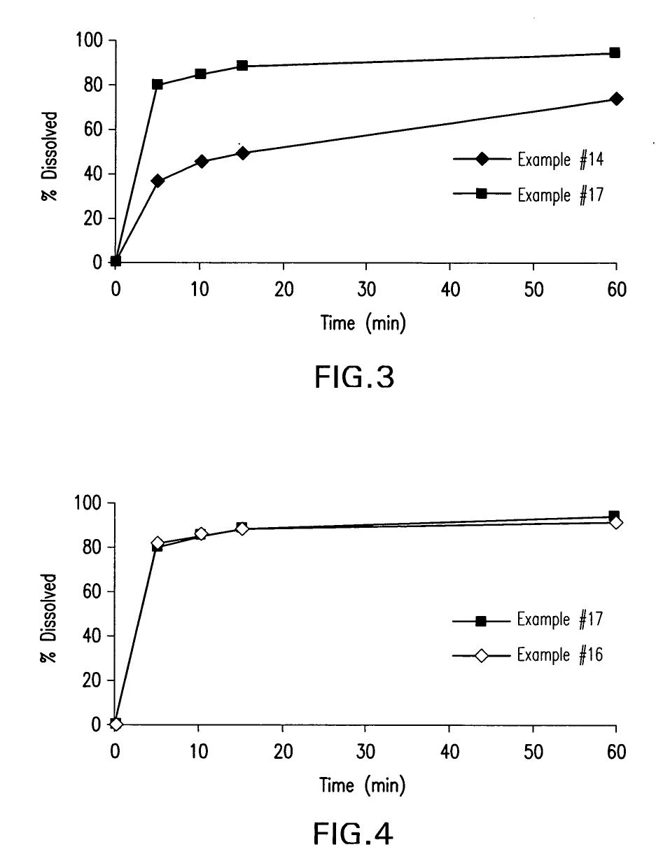

Table 3 below shows the fexofenadine HCl dissolution profiles of tablet formulation Examples 14 and 17, which is plotted in FIG. 3. Example 14 uses crosprovidone as a disintegrant and Example 17 uses sodium starch glycolate as a disintegrant. These results demonstrate that the release profile of fexofenadine HCl is affected by the type of disintegrant in the tablet formulation. The use of sodium starch glycolate (Example 17) as a disintegrant results in a higher dissolution profile for fexofenadine HCl in comparison to the use of crospovidone (Example 14) as a disintegrant. Therefore, in certain embodiments, sodium starch glycolate is a preferred disintegrant.

TABLE 3
% Dissolution of Fexofenadine HCl

| Time | Example | | Example | |
|---|---|---|---|---|
| (min) | 14 | RSD | 17 | RSD |
| 0 | 0 | 0 | 0 | 0 |
| 5 | 37 | 29.1 | 80 | 0.3 |
| 10 | 45 | 25.0 | 85 | 0.5 |
| 15 | 50 | 24.6 | 88 | 1.7 |
| 60 | 74 | 16.5 | 94 | 2.2 |

Table 4 below shows the fexofenadine HCl dissolution profiles of tablet formulation Examples 16 and 17, which is plotted in FIG. 4. Example 16 uses 5% of sodium starch glycolate and Example 17 uses 2.5%. These results demonstrate the effect of different amounts of disintegrant in the tablet formulation. In certain embodiments, using 2.5% of sodium starch glycolate as the disintegrant in the tablet formulation is preferred.

TABLE 4
% Dissolution of Fexofenadine HCl

| Time | Example | | Example | |
|---|---|---|---|---|
| (min) | 16 | RSD | 17 | RSD |
| 0 | 0 | 0 | 0 | 0 |
| 5 | 82 | 1.5 | 80 | 0.3 |
| 10 | 86 | 3.5 | 85 | 0.5 |
| 15 | 88 | 3.6 | 88 | 1.7 |
| 60 | 92 | 4.6 | 94 | 2.2 |

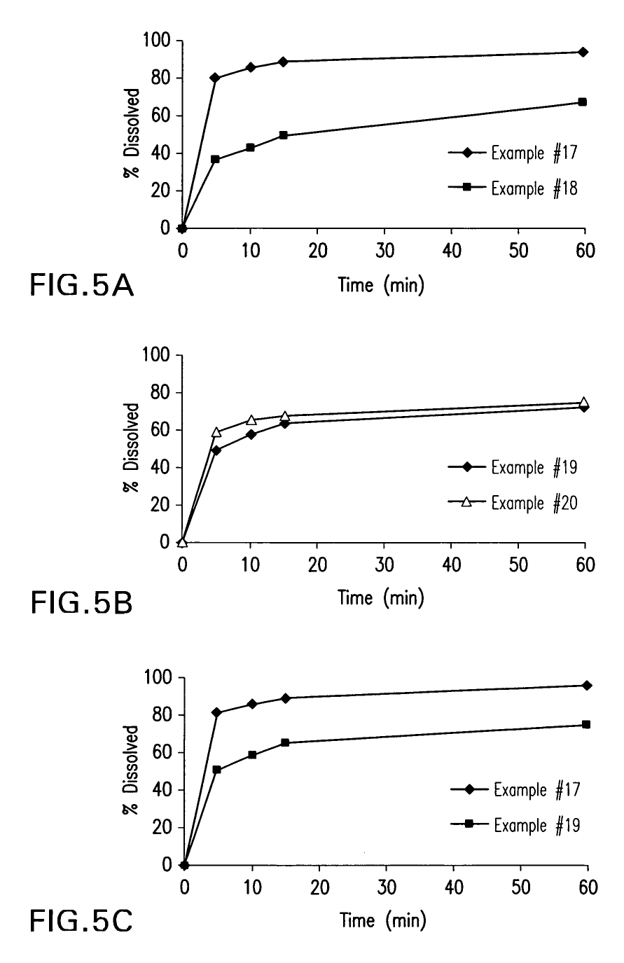

Table 5A below shows the fexofenadine HCl dissolution profiles of tablet formulation Examples 17 and 18, which is plotted in FIG. 5A. Table 5B below shows the fexofenadine HCl dissolution profiles of tablet formulation Examples 19 and 20, which is plotted in FIG. 5B. Table 5C below shows the fexofenadine HCl dissolution profiles of tablet formulation Examples 17 and 19, which is plotted in FIG. 5C. Examples 17 and 19 use Avicel PH 101™ (microcrystalline cellulose) as a filler; Example 18 uses Mannitol Parteck™ as a filler; and Example 20 uses Lactose Spray Dried™ as a filler.

It is important to note that in Examples 19 and 20, sodium lauryl sulfate is added. This anionic surfactant reduces the dissolution rate of fexofenadine HCl as can be determined by comparing the profiles of Examples 17 and 19 (Table 5C), which both contain Avicel PH101™ in their formulation. In some embodiments, microcrystalline cellulouse (such as Avicel PH101) is a preferred filler because it allows for an immediate release of the fexofenadine HCl.

TABLE 5A
% Dissolution of Fexofenadine HCl

| Time | Example | | Example | |
|---|---|---|---|---|
| (min) | 17 | RSD | 18 | RSD |
| 0 | 0 | 0 | 0 | 0 |
| 5 | 80 | 0.3 | 37 | 32.3 |
| 10 | 85 | 0.5 | 43 | 21.6 |
| 15 | 88 | 1.7 | 49 | 16.1 |
| 60 | 94 | 2.2 | 67 | 2.8 |

TABLE 5B
% Dissolution of Fexofenadine HCl

| Time | Example | | Example | |
|---|---|---|---|---|
| (min) | 19 | RSD | 20 | RSD |
| 0 | 0 | 0 | 0 | 0 |
| 5 | 50 | 13.1 | 60 | 2.3 |
| 10 | 58 | 8.1 | 66 | 1.7 |
| 15 | 64 | 3.4 | 68 | 1.6 |
| 60 | 73 | 5.7 | 75 | 1.7 |

TABLE 5C
% Dissolution of Fexofenadine HCl

| Time | Example | | Example | |
|---|---|---|---|---|
| (min) | 17 | RSD | 19 | RSD |
| 0 | 0 | 0 | 0 | 0 |
| 5 | 80 | 0.3 | 50 | 13.1 |
| 10 | 85 | 0.5 | 58 | 8.1 |
| 15 | 88 | 1.7 | 64 | 3.4 |
| 60 | 94 | 2.2 | 73 | 5.7 |

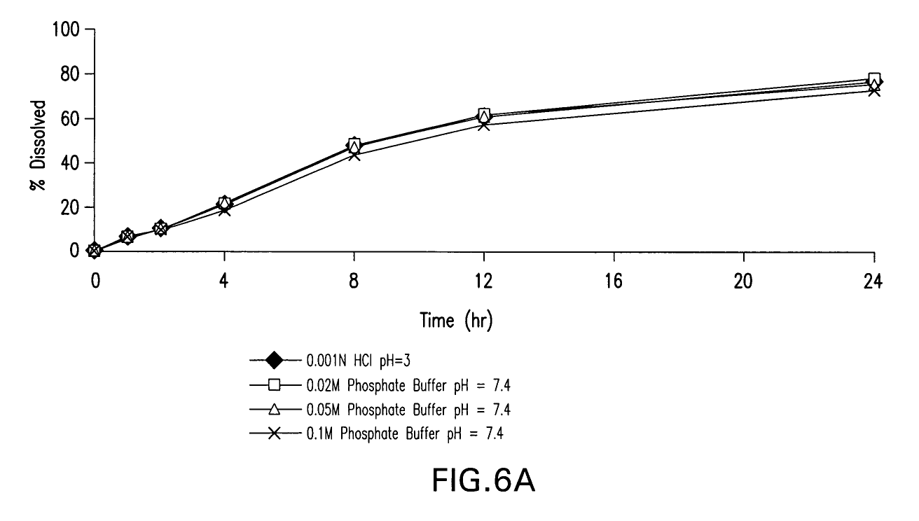

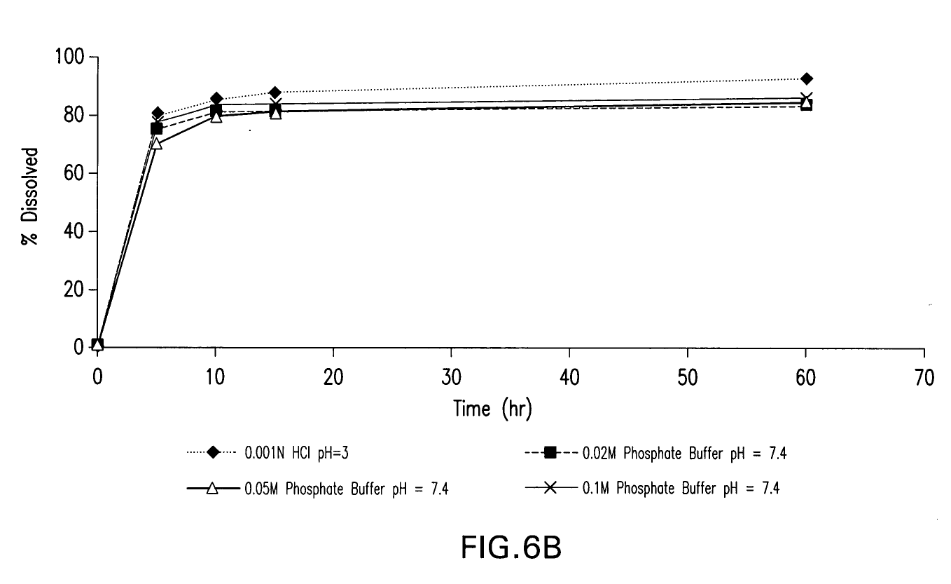

The dissolution profiles of the pseudoephedrine HCl and fexofenadine HCl in formulation Example 17 were examined in different dissolution mediums which differed by their pH and ionic strength. Table 6A below shows the pseudoephedrine HCl dissolution profile of Example 17 in various mediums, which is plotted in FIG. 6A. Table 6B below shows the fexofenadine HCl dissolution profile for Example 17 in various mediums, which is plotted in FIG. 6B. These results suggest that the dissolution profile of both drug substances as exemplified in Example 17 are not significantly affected by different dissolution mediums.

TABLE 6A
% Dissolution of Pseudoephedrine HCl

| | | | 0.02M | | 0.05M | | 0.1M | |
|---|---|---|---|---|---|---|---|---|
| | 0.001N | | Phosphate | | Phosphate | | Phosphate | |
| Time | HCl | | Buffer | | Buffer | | Buffer | |
| (hr) | pH = 3 | RSD | pH = 7.4 | RSD | pH = 7.4 | RSD | pH = 7.4 | RSD |
| 0 | 0 | 0 | 0 | 0 | 0 | 0 | 0 | 0 |
| 1 | 7 | 3.5 | 7 | 13.2 | 7 | 63.9 | 6 | 10.3 |
| 2 | 10 | 0.9 | 11 | 13.8 | 11 | 57.3 | 10 | 6.7 |
| 4 | 22 | 1.5 | 21 | 6.1 | 21 | 38.8 | 18 | 2.4 |
| 8 | 48 | 0.4 | 48 | 0.0 | 47 | 19.5 | 43 | 0.2 |
| 12 | 61 | 0.9 | 62 | 0.7 | 61 | 16.7 | 57 | 0.5 |
| 24 | 76 | 1 | 77 | 0.2 | 75 | 13.0 | 72 | 0.0 |

TABLE 6B
% Dissolution of Fexofenadine HCl

| | | | 0.02M | | 0.05M | | 0.1M | |
|---|---|---|---|---|---|---|---|---|
| | 0.001N | | Phosphate | | Phosphate | | Phosphate | |
| Time | HCl | | Buffer | | Buffer | | Buffer | |
| (min) | pH = 3 | RSD | pH = 7.4 | RSD | pH = 7.4 | RSD | pH = 7.4 | RSD |
| 0 | 0 | 0 | 0 | 0 | 0 | 0 | 0 | 0 |
| 5 | 80 | 0.3 | 75 | 0.1 | 70 | 13.7 | 78 | 0.6 |
| 10 | 85 | 0.5 | 81 | 1.7 | 80 | 0.4 | 84 | 3.4 |
| 15 | 88 | 1.7 | 82 | 1.1 | 81 | 1.5 | 84 | 2.4 |
| 60 | 94 | 2.2 | 84 | 0.3 | 85 | 4.4 | 87 | 1.8 |

1. A pharmaceutical composition comprising:
(a) a mixture comprising a first therapeutic agent; and
(b) a plurality of particles disposed within the mixture, wherein the particles comprise
(i) an interior comprising a second therapeutic agent, and
(ii) an exterior comprising a material for controlling the release of the second therapeutic agent.

2. The composition of claim 1, wherein the interior comprises an inner core and an intermediate layer disposed over the inner core, and wherein the inner core comprises a pharmaceutically inactive material, and wherein the intermediate layer comprises the second therapeutic agent.

3. The composition of claim 2, wherein at least 85% of the inner cores have a size in the range of 100 to 1000 μm.

4. The composition of claim 3, wherein at least 85% of the inner cores have a size in the range of 100 to 850 μm.

5. The composition of claim 4, wherein at least 85% of the inner cores have a size in the range of 100 to 710 μm.

6. The composition of claim 5, wherein at least 85% of the inner cores have a size in the range of 100 to 500 μm.

7. The composition of claim 6, wherein at least 85% of the inner cores have a size in the range of 100 to 425 μm.

8. The composition of claim 7, wherein at least 85% of the inner cores have a size in the range of 100 to 355 μm.

9. The composition of claim 8, wherein at least 85% of the inner cores have a size in the range of 200 to 355 μm.

10. The composition of claim 2, wherein the inner cores comprise microcrystalline cellulose spheres or sugar spheres.

11. The composition of claim 1, wherein the interior further comprises a binder.

12. The composition of claim 11, wherein the binder is selected from the group consisting of: polyvinyl pyrrolidone (PVP), methyl cellulose, hydroxypropyl cellulose, and hydroxypropyl methylcellulose

13. The composition of claim 12, wherein the binder is polyvinyl pyrrolidone (PVP).

14. The composition of claim 1, wherein the material for controlling the release of the second therapeutic agent comprises a polymeric material.

15. The composition of claim 1, wherein the exterior further comprises one or more plasticizing agents.

16. The composition of claim 15, wherein the one or more plasticizing agents include a hydrophilic plasticizer.

17. The composition of claim 1, wherein the first therapeutic agent comprises a piperidinoalkanol and the second therapeutic agent comprises a decongestant.

18. The composition of claim 17, wherein the composition is in an oral dosage form.

19. The composition of claim 18, wherein the composition is in the form of a tablet.

20. (canceled)

21. The composition of claim 17, wherein the decongestant is formulated for extended release.

22. The composition of claim 21, wherein less than 60% of the decongestant is released in a time period of 8 hours after exposure of the oral dosage form to an aqueous solution.

23. The composition of claim 1, wherein the mixture further comprises one or more pharmaceutically acceptable excipients.

24. The composition of claim 23, wherein the mixture includes microcrystalline cellulose, sodium starch, or both.

25. The composition of claim 17, wherein the piperidinoalkanol is formulated for immediate release.

26. The composition of claim 25, wherein at least 50% of the piperidinoalkanol is released within 15 minutes after exposure of the oral dosage form to an aqueous solution.

27. The composition of claim 26, wherein at least 75% of the piperidinoalkanol is released within 15 minutes after exposure of the oral dosage form to an aqueous solution.

28. The composition of claim 17, wherein the piperidinoalkanol is fexofenadine or a pharmaceutically acceptable salt thereof.

29. The composition of claim 17, wherein the decongestant is pseudoephedrine or a pharmaceutically acceptable salt thereof.

30. A method for making a pharmaceutical composition, comprising the steps of:
(a) forming a plurality of particles, comprising the steps of:
(i) providing an inner core,
(ii) disposing an intermediate layer containing a second therapeutic agent over the inner core, and
(iii) disposing an extended release layer over the intermediate layer, wherein the extended release layer comprises a material for controlling the release of the second therapeutic agent; and
(b) combining the particles with a mixture comprising a first therapeutic agent.

31-42. (canceled)

43. A method for making a pharmaceutical composition, comprising the step of combining:
(a) a plurality of particles comprising
(i) an interior comprising a second therapeutic agent, and
(ii) an exterior comprising a material for controlling the release of the second therapeutic agent; and
(b) a mixture comprising a first therapeutic agent.

44-64. (canceled)

65. A method of treating congestion in a patient in need thereof by administering to the patient a pharmaceutical composition comprising:
(a) a mixture comprising a piperidinoalkanol; and
(b) a plurality of particles comprising,
(i) an interior comprising a decongestant, and
(ii) an exterior comprising a material for controlling the release of the decongestant.

66-92. (canceled)

93. A pharmaceutical composition comprising:
(a) a dry blend comprising a piperidinoalkanol; and
(b) a plurality of particles comprising,
(i) an interior comprising a decongestant, and
(ii) an exterior comprising a material for controlling the release of the decongestant.

94. (canceled)

95. A method for making a pharmaceutical composition, comprising the steps of:
(a) forming a plurality of particles, comprising the steps of:
(i) providing an inner core,
(ii) disposing an intermediate layer containing a decongestant over the inner core, and
(iii) disposing an extended release layer over the intermediate layer, wherein the extended release layer comprises a material for controlling the release of the decongestant; and
(b) mixing the particles with a dry blend comprising a piperidinoalkanol to form a mixture.

96. A method for making a pharmaceutical composition, comprising the step of combining:
(a) a plurality of particles comprising:
(i) an interior comprising a decongestant, and
(ii) an exterior comprising a material for controlling the release of the decongestant; and,
(b) a dry blend comprising a piperidinoalkanol.

97. (canceled)
